(19) United States
(12) Patent Application Publication
(10) Pub. No.: US 2024/0219367 A1
(43) Pub. Date: Jul. 4, 2024

(54) Determining a Frequency for Tumor Treating Fields (TTFields) Therapy Based on Tests Performed on the Tumor Cells

(71) Applicant: Novocure GmbH, Root D4 (CH)

(72) Inventors: Yoram WASSERMAN, Haifa (IL); Mor BEN-TOV KUPERBERG, Haifa (IL)

(73) Assignee: Novocure GmbH, Root D4 (CH)

(21) Appl. No.: 18/398,517

(22) Filed: Dec. 28, 2023

Related U.S. Application Data
(60) Provisional application No. 63/435,900, filed on Dec. 29, 2022.

(51) Int. Cl. G01N 33/483; A61N 1/36

(52) U.S. Cl. CPC G01N 33/4836; A61N 1/36002; G01N 2800/52

(57) ABSTRACT

Cancer treatment using TTFields (Tumor Treating Fields) can be customized to each individual subject by extracting cancer cells from the subject's body. Alternating electric fields are then applied to the extracted cells at different frequencies, and voltage measurements are obtained from the cells under two different conditions at each of the different frequencies. These voltage measurements can then be used to determine what frequency will provide the largest gradient at the cleavage furrow when similar cells divide, which will in turn increase the efficacy of the TTFields. Treatment using TTFields can then proceed at the determined frequency.

### CROSS REFERENCE TO RELATED APPLICATIONS

This application claims the benefit of U.S. Provisional Application 63/435,900, filed Dec. 29, 2022, which is incorporated herein by reference in its entirety.

### BACKGROUND

Tumor Treating Fields (TTFields) therapy is a proven approach for treating tumors using alternating electric fields at frequencies between 50 kHz and 1 MHz (e.g., 150-250 kHz). TTFields therapy has received FDA approval for treating Glioblastoma Multiforme (GBM) brain tumors and appears to be very promising for many other types of tumors. The Optune® system delivers TTFields to patients via four transducer arrays that are placed on the patient's skin near the tumor. The transducer arrays are arranged in two pairs, with one pair of transducer arrays positioned to the left and right of the tumor, and the other pair of transducer arrays positioned anterior and posterior to the tumor. An AC signal generator (a) sends an AC current through the anterior/posterior pair of transducer arrays for 1 second, which induces an electric field with a first direction through the tumor; then (b) sends an AC current through the left/right pair of arrays for 1 second, which induces an electric field with a second direction through the tumor; then repeats steps (a) and (b) for the duration of the treatment.

Traditionally, the frequency at which the TTFields were applied to the subject was based on the particular type of tumor that was being treated. For example, 200 kHz is the recommended frequency for TTFields when treating GBM; and 150 kHz is the recommended frequency for TTFields when treating gastric cancer.

### SUMMARY OF THE INVENTION

One aspect of the invention is directed to a first method of determining a frequency for treating a particular subject using an alternating electric field. The first method comprises electrically connecting a first electrode to an interior of a first cancer cell obtained from the subject, electrically connecting a second electrode to an interior of a second cancer cell obtained from the subject, electrically connecting a third electrode to the interior of the first cancer cell, and electrically connecting a fourth electrode to the interior of the second cancer cell. The first method also comprises applying an external alternating electric field to the first and second cancer cells at a plurality of different frequencies at a respective plurality of different times, and measuring a respective first voltage between the third electrode and the fourth electrode while the external alternating electric field is being applied at each of the plurality of different frequencies during times when a low resistance path is established between the first electrode and the second electrode. The first method also comprises measuring a respective second voltage between the third electrode and the fourth electrode while the external alternating electric field is being applied at each of the plurality of different frequencies during times when a low resistance path is not established between the first electrode and the second electrode, and determining a frequency for treating the subject using an alternating electric field based at least in part on the measured first voltages and the measured second voltages.

In some instances of the first method, the determining comprises selecting a frequency at which a difference between the respective first voltage and the respective second voltage is largest. In some instances of the first method, the determining comprises selecting a frequency at which a difference between the respective first voltage and the respective second voltage is within 10% of the largest difference. In some instances of the first method, the determining comprises selecting a frequency at which a difference between the respective first voltage and the respective second voltage is within 25% of the largest difference.

In some instances of the first method, each of the plurality of different frequencies is between 50 kHz and 1 MHz. In some instances of the first method, each of the plurality of different frequencies is between 75 kHz and 500 kHz. In some instances of the first method, the first electrode comprises a hollow glass micropipette filled with a conductive liquid, and the second electrode comprises a hollow glass micropipette filled with a conductive liquid.

In some instances of the first method, each of the plurality of different frequencies is between 75 kHz and 500 kHz, and the determining comprises selecting a frequency at which a difference between the respective first voltage and the respective second voltage is within 25% of the largest difference.

Some instances of the first method further comprise obtaining the first cancer cell from the subject, obtaining the second cancer cell from the subject, and treating the subject using an alternating electric field at the determined frequency.

Another aspect of the invention is directed to a first apparatus for determining a frequency for treating a particular subject using an alternating electric field. The first apparatus comprises a first electrode configured to electrically connect with an interior of a first cell, a second electrode configured to electrically connect with an interior of a second cell, and a switch. The switch has a first terminal electrically connected to the first electrode and also has a second terminal electrically connected to the second electrode. The switch can operate in either (a) a closed state that establishes a low resistance path between the first electrode and the second electrode or (b) an open state that does not establish a low resistance path between the first electrode and the second electrode.

In some embodiments of the first apparatus, the first electrode comprises a hollow glass micropipette filled with a conductive liquid, and the second electrode comprises a hollow glass micropipette filled with a conductive liquid.

Some embodiments of the first apparatus further comprise the first cell and the second cell. In these embodiments, the first electrode is electrically connected with the interior of the first cell, and the second electrode is electrically connected with the interior of the second cell.

Optionally, the embodiments described in the previous paragraph may further comprise a third electrode and a fourth electrode. The third electrode is configured to electrically connect with the interior of the first cell, and the third electrode is electrically connected with the interior of the first cell. The fourth electrode is configured to electrically connect with the interior of the second cell, and the fourth electrode is electrically connected with the interior of the second cell.

Optionally, the embodiments described in the previous paragraph may further comprise a controller configured to control an electric field generator and the state of the switch so that while the switch is in the closed state, the electric field generator applies an electric field at a plurality of different frequencies to the first cell and to the second cell at respective different first times, wherein each of the plurality of different frequencies is between 50 kHz and 1 MHz. In these embodiments, the controller is also configured to input a respective first voltage measurement obtained using the third electrode and the fourth electrode during each of the first times. The controller is also configured to control the electric field generator and the state of the switch so that while the switch is in the open state, the electric field generator applies an electric field at the plurality of different frequencies to the first cell and to the second cell at respective different second times. And the controller is also configured to input a respective second voltage measurement obtained using the third electrode and the fourth electrode during each of the second times.

Optionally, in the embodiments described in the previous paragraph, the controller may be further configured to determine a frequency that maximizes a difference between a respective first voltage measurement and a respective second voltage measurement.

Another aspect of the invention is directed to a second method of determining a frequency for treating a particular subject using an alternating electric field. The second method comprises electrically connecting a first electrode to an interior of a first cell obtained from the subject, electrically connecting a second electrode to an interior of a second cell obtained from the subject, electrically connecting a third electrode to the interior of the first cell, and electrically connecting a fourth electrode to the interior of the second cell. The second method also comprises applying an external alternating electric field to the first and second cells, and measuring a first voltage between the third electrode and the fourth electrode while the external alternating electric field is being applied during at least one time when a low resistance path is established between the first electrode and the second electrode. The second method also comprises measuring a second voltage between the third electrode and the fourth electrode while the external alternating electric field is being applied during at least one time when a low resistance path is not established between the first electrode and the second electrode, and determining a frequency for treating the subject using an alternating electric field based at least in part on the measured first voltage and the measured second voltage.

Some instances of the second method further comprise obtaining the first cell from the subject and obtaining the second cell from the subject.

Another aspect of the invention is directed to a third method of measuring a characteristic of an electric field in an interior of a cell. The third method comprises electrically connecting a first electrode to the interior of the cell via the cell membrane, electrically connecting a second electrode to the interior of the cell via the cell membrane so that a distal tip of the second electrode is spaced apart from a distal tip of the first electrode, applying an external alternating electric field to the cell, and measuring a voltage between the first electrode and the second electrode while the external alternating electric field is being applied to the cell.

In some instances of the third method, the external electric field has a frequency between 50 kHz and 1 MHz. In some instances of the third method, the external electric field has a frequency between 75 kHz and 500 kHz. In some instances of the third method, the first electrode comprises a hollow glass micropipette filled with a conductive liquid, and the second electrode comprises a hollow glass micropipette filled with a conductive liquid.

Another aspect of the invention is directed to a fourth method of determining a frequency of an alternating electric field that will maximize an electric gradient when cells within a given population divide. The fourth method comprises electrically connecting a first electrode to an interior of a first cell obtained from the population, electrically connecting a second electrode to an interior of a second cell obtained from the population, electrically connecting a third electrode to the interior of the first cell, and electrically connecting a fourth electrode to the interior of the second cell. The fourth method also comprises applying an external alternating electric field to the first and second cells at a plurality of different frequencies at a respective plurality of different times, and measuring a respective first voltage between the third electrode and the fourth electrode while the external alternating electric field is being applied at each of the plurality of different frequencies during times when a low resistance path is established between the first electrode and the second electrode. The fourth method also comprises measuring a respective second voltage between the third electrode and the fourth electrode while the external alternating electric field is being applied at each of the plurality of different frequencies during times when a low resistance path is not established between the first electrode and the second electrode, and determining a frequency for applying an alternating electric field to the population of cells based at least in part on the measured first voltages and the measured second voltages.

In some instances of the fourth method, the determining comprises selecting a frequency at which a difference between the respective first voltage and the respective second voltage is largest. In some instances of the fourth method, the determining comprises selecting a frequency at which a difference between the respective first voltage and the respective second voltage is within 10% of the largest difference. In some instances of the fourth method, the determining comprises selecting a frequency at which a difference between the respective first voltage and the respective second voltage is within 25% of the largest difference.

In some instances of the fourth method, each of the plurality of different frequencies is between 50 kHz and 1 MHz. In some instances of the fourth method, each of the plurality of different frequencies is between 75 kHz and 500 kHz. In some instances of the fourth method, the first electrode comprises a hollow glass micropipette filled with a conductive liquid, and the second electrode comprises a hollow glass micropipette filled with a conductive liquid.

In some instances of the fourth method, each of the plurality of different frequencies is between 75 kHz and 500 kHz, and the determining comprises selecting a frequency at which a difference between the respective first voltage and the respective second voltage is within 25% of the largest difference.

### BRIEF DESCRIPTION OF THE DRAWINGS

FIG. 1 is a schematic representation of TTFields being applied to a dividing cell at a time just before the original parent cell divides into two daughter cells.

FIG. 2 is a schematic diagram of an apparatus for determining a frequency for treating a particular subject using TTFields.

FIG. 3 is a flowchart for controlling the switch and the electric field generator of FIG. 2 in order to determine the frequency for treating a particular subject using TTFields.

FIG. 4. depicts two separate cells that reside in a conductive medium.

FIG. 5 depicts a different approach that directly measures the voltage gradient inside a cell.

Various embodiments are described in detail below with reference to the accompanying drawings, wherein like reference numerals represent like elements.

### DESCRIPTION OF THE PREFERRED EMBODIMENTS

While the traditional approach of using the same frequency for all subjects with a given type of cancer is a workable solution, using a single frequency for all subjects may not provide the best results in each and every one of those subjects. For example, while 200 kHz may be the best frequency for most subjects with GBM, certain individuals with GBM might respond better to a different frequency (e.g., 175 kHz or 225 kHz).

TTFields do not damage nondividing cells, and only have an effect on cells while the cells are dividing. And because cancer cells divide much more rapidly than normal cells, TTFields selectively target cancer cells.

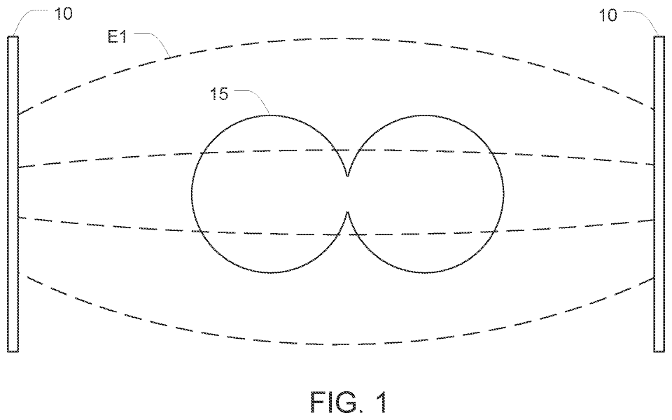

FIG. 1 is a schematic representation of TTFields E1 being applied to a dividing cell 15 at a time just before the original parent cell divides into two daughter cells. At this stage, the dividing parent cell has a shape that resembles an hourglass in that it has two portions connected by a relatively narrow neck (i.e., the cleavage furrow). The TTFields E1 are generated by applying a voltage across a pair of electrodes 10.

While not being bound by this theory, it is believed that increasing the gradient of the electric field at the cleavage furrow increases the efficacy of TTFields. Two competing factors contribute to increasing the gradient of the electric field at the cleavage furrow of the dividing cell 15. The first factor deals with getting the electric field to enter the dividing cell. Because cell membranes have a capacitance, the impedance of the cell membrane is inversely proportional to frequency. Therefore, when a dividing cell is positioned within a conductive material and TTFields are applied to the conductive material, it will be easier for TTFields with higher frequencies to cross the cell membrane than TTFields with lower frequencies. The first factor therefore favors the use of higher frequencies.

The second factor relates to the path that the field lines of the TTFields take after the TTFields has entered the dividing cell. At the state depicted in FIG. 1, the path between the two daughter cells is a low resistive-impedance path, with virtually zero capacitive components in that path. At lower frequencies, the material within the dividing cell will have a lower impedance than the cell membrane, in which case the current will preferentially follow the lower impedance path through the cleavage furrow. But as the frequency of the TTFields increases to a point where the impedance of the cell membrane drops far enough, the current's preference to follow the path through the cleavage furrow disappears. The second factor therefore favors the use of lower frequencies.

*IF* it was easy to directly measure the gradient of the electric field at the cleavage furrow of a dividing cell, determining which frequency balances the two factors discussed above to provide the largest gradient would be straightforward. For *IF* it was easy to directly measure the gradient, a dividing cell could be positioned within a conductive material (e.g., saline), and an alternating electric field could be applied to the conductive material at many different frequencies. The resulting gradient at the cleavage furrow could then be measured for each of the different frequencies, and the largest gradient could be identified. But in practice, directly measuring the gradient of the electric field at the cleavage furrow of a dividing cell is exceedingly difficult, if not impossible.

This application discloses an indirect approach for determining which frequency balances the two factors discussed above to maximize the gradient at the cleavage furrow of a dividing cell (which will, in turn, increase the efficacy of TTFields). This approach can advantageously be used to select an individualized frequency of TTFields treatment for each individual subject. And notably, it relies on testing of cancer cells that have been extracted from an individual subject before TTFields therapy for that same individual subject begins.

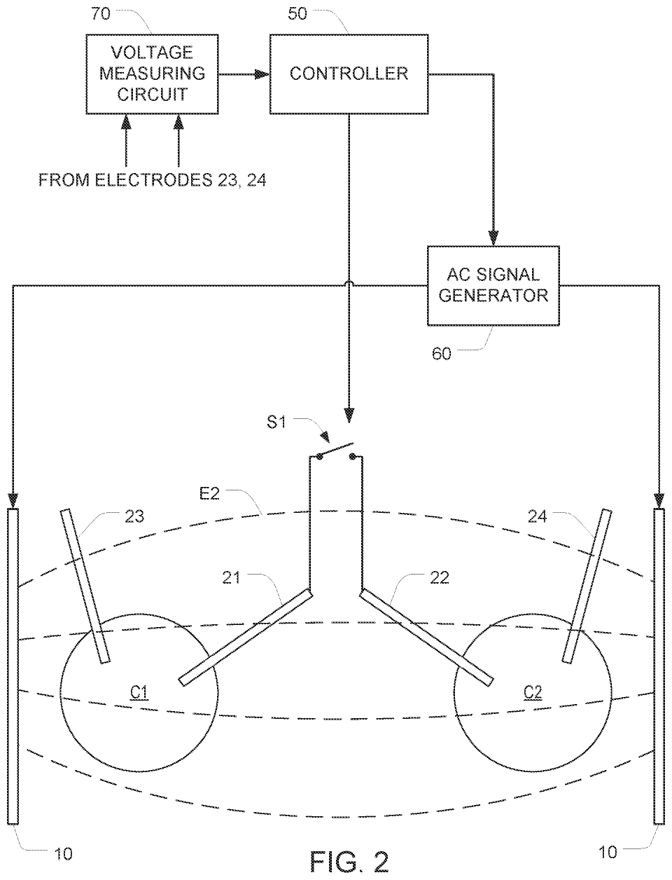

FIG. 2 is a schematic diagram of an apparatus for determining a frequency for treating a particular subject using an alternating electric field (e.g., TTFields). First and second cancer cells C1, C2 are extracted (e.g., during a biopsy) from the particular subject's body who will eventually be treated.

First and second electrodes 21, 22 are configured to electrically connect with the interior of first and second cells C1, C2, respectively. Each of these electrodes 21, 22 can be implemented using a hollow glass micropipette filled with a conductive liquid (e.g., saline) e.g., similar to conventional patch clamp electrodes. Alternative approaches for implementing these electrodes 21, 22 can also be used. The first electrode 21 is electrically connected with the interior of the first cell C1 and the second electrode 22 is electrically connected with the interior of the second cell C2 e.g., using techniques similar to conventional patch clamp electrodes.

A switch S1 has one terminal that is electrically connected to the first electrode 21 and a second terminal that is electrically connected to the second electrode 22. The switch S1 can operate in either (a) a closed state that establishes a low resistance path between the first electrode and the second electrode or (b) an open state that does not establish a low resistance path between the first electrode and the second electrode.

Third and fourth electrodes 23, 24 are configured to electrically connect with the interior of the first and second cells C1, C2, respectively. Each of these electrodes 23, 24 can be implemented as described above for the first and second electrodes 21, 22. The third electrode 23 is electrically connected with the interior of the first cell C1 and the fourth electrode 24 is electrically connected with the interior of the second cell C2 (e.g., using the same techniques described above for the first and second electrodes 21, 22).

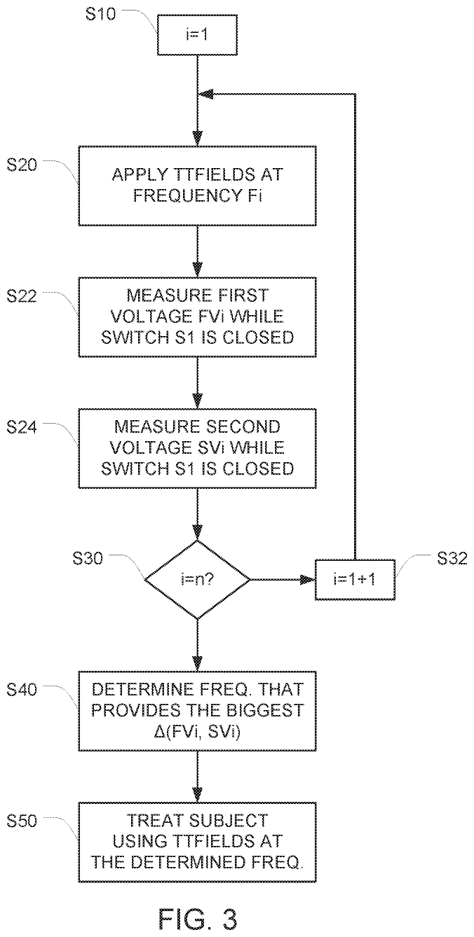

A controller 50 is configured to control the state of the switch S1 and an electric field generator (which, in the example embodiment illustrated in FIG. 2, includes an AC signal generator 60 connected to electrodes 10 that are arranged and positioned to impose an AC field E2 in the medium in which the cells C1, C2 reside). The state of the switch S1 and the electric field generator are controlled as described below in connection with FIG. 3. The FIG. 2 embodiment also includes a voltage measuring circuit 70 that is configured to measure the AC voltage that is induced across the third and fourth electrodes 23, 24 when an electric field E2 is applied to the cells C1, C2 under various different conditions. The voltage measuring circuit 70 could be, e.g., a voltmeter, an analog front end configured to measure voltages, an oscilloscope, or any of a variety of other voltage measuring circuits.

FIG. 3 is a flowchart that shows how the controller 50 controls the state of the switch S1 and the electric field generator 60 +10 in order to determine the frequency for treating a particular subject using an alternating field (e.g., TTFields). Processing begins at S10 where the controller initializes the loop counter variable i to 1.

At S20, the controller 50 commands the AC signal generator 60 to output a first frequency F1, which applies an alternating electric field to the first and second cells at a first time via the electrodes 10.

At S22, the controller 50 commands the switch S1 to close and subsequently inputs a first voltage measurement FV1 via the voltage measuring circuit 70. This first voltage measurement FV1 represents the voltage between the third and fourth electrodes 23, 24 while an alternating electric field is applied to the cells C1, C2 at the first frequency while the switch S1 is closed. Then, at S24, the controller 50 commands the switch S1 to open and subsequently inputs a second voltage measurement SV1 via the voltage measuring circuit 70. This second voltage measurement SV1 represents the voltage between the third and fourth electrodes 23, 24 while an alternating electric field is applied to the cells C1, C2 at the first frequency while the switch S1 is open.

Steps S30 and S32 collectively determine if additional passes through the loop are needed and initiate any such additional passes through the loop. More specifically, at S30, the controller 50 checks to see whether the loop counter i has reached its maximum value n. If the loop counter has not reached its maximum value, the loop counter i is incremented in S32, and processing returns to S20 for an additional pass (e.g., a 2nd, 3rd, 4th, . . . nth pass) through the loop S20-S24 at the next frequency. Alternatively, if the loop counter i has reached its maximum value n, processing proceeds to step S40. By using this loop structure, the controller 50 obtains a first voltage measurement FVi (while S1 is closed) and a second voltage measurement SVi (while S1 is open) at each of the different frequencies (i.e., F1 through Fn).

In the special case where the maximum value for the loop counter is 1 (i.e., n=1), there will only be a single pass through the loop. There will therefore be only a single first voltage and a single second voltage.

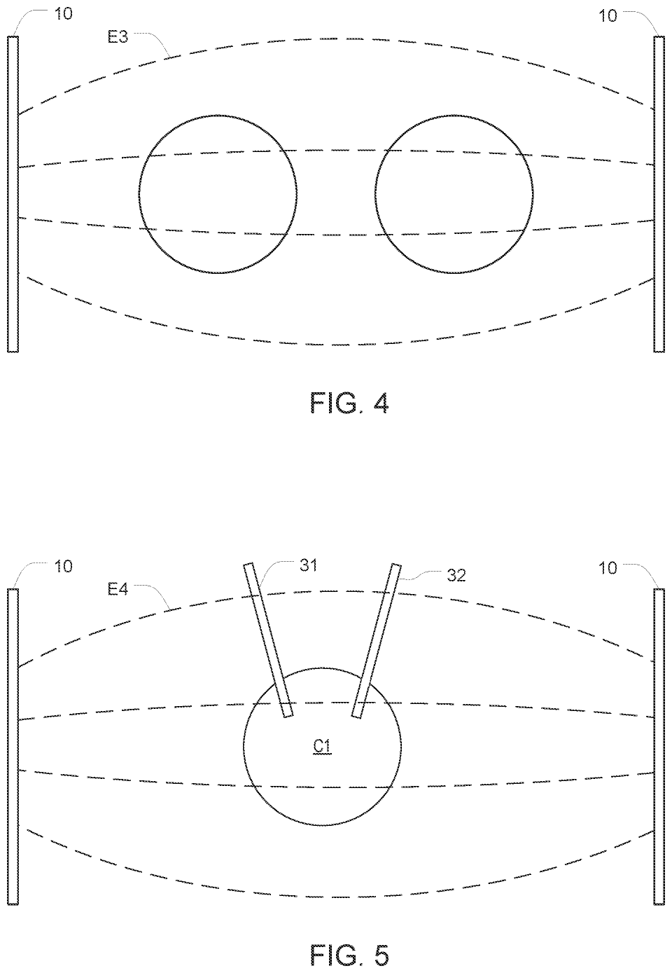

Notably, because closing switch S1 establishes a low impedance path between the first and second electrode 21, 22 (which are respectively connected to the interior of the first and second cells C1, C2), closing switch S1 simulates the situation depicted in FIG. 1 in which two daughter cells are connected via a low resistive-impedance path. On the other hand, opening switch S1 simulates the situation in which two separate cells reside in a conductive medium, as depicted in FIG. 4.

After collecting the voltage measurements FVi and SVi (for i=1 to n) obtained as described above in connection with steps S22 and S24 (implemented at n different frequencies), those measurements can be used to predict the gradient at the interconnection between the two daughter cells during mitosis. More specifically, the magnitude of that gradient is related to the difference between (a) the voltage measured using the third and fourth electrodes 23, 24 in FIG. 2 when an alternating electric field is applied at the given frequency while the switch S1 is closed and (b) the voltage measured using those same electrodes 23, 24 when an alternating electric field is applied at the same given frequency while the switch S1 is open.

The measured first voltages (FV1-FVn) and the measured second voltages (SV1-SVn) can therefore be used in S40 to determine the frequency for treating the subject using an alternating electric field. This can be accomplished, e.g., by selecting the frequency at which a difference between the respective first voltage (FVi) and the respective second voltage (SVi) is largest, for i=1 to n. Alternatively, it can be accomplished by selecting a frequency at which a difference between the respective first voltage (FVi) and the respective second voltage (SVi) is within 1%, 2%, 5%, 10%, or 25% of the largest difference.

The number of frequencies tested (i.e., n) can range from 2-100 or even higher. For example, if frequencies between 75 kHz and 500 kHz are tested, and the tested frequencies are spaced apart by 25 kHz, n would be 18. In another example, if frequencies between 190 kHz and 210 kHz are tested, and the tested frequencies are spaced apart by 1 kHz, n would be 21. Each of the plurality of different frequencies can be, e.g., between 50 kHz and 1 MHz, between 75 kHz and 500 kHz, between 75 kHz and 300 kHz, etc. Narrower ranges (e.g., between 175 kHz and 225 kHz for a glioblastoma patient) can be used when the expected range of frequencies can be predicted based on the type of cancer being treated.

Advantageously, the techniques described above in connection with FIGS. 2 and 3 do not require precise alignment of the distal tips of the first, second, third, and fourth electrodes 21-24, and do not require specific spacings between those electrodes. In fact, the techniques described above are agnostic to the spacing between the electrode elements, and can be used to determine a frequency for treatment even when the electrodes' spacings vary widely. Furthermore, the techniques described above are agnostic to the amplitude of the alternating electric fields that are applied to the cells C1, C2 (as long as the same amplitude of the applied electric field E2 is used for all the frequencies that are tested). This is because the difference-in-voltage technique described above does not provide a direct measurement of the gradient at the cleavage furrow. Instead, it provides indirect data, and information about the frequency that yields the largest gradient can be inferred from that indirect data.

After a frequency has been determined in S40, the subject is treated using TTFields at that frequency in S50.

FIG. 5 depicts a different approach that directly measures the voltage gradient inside a cell. In this approach, a first electrode 31 is electrically connected to the interior of the cell C1 via the cell membrane, and a second electrode 32 is electrically connected to the interior of the cell via the cell membrane so that a distal tip of the second electrode is spaced apart from a distal tip of the first electrode. For example, the distal tip of the second electrode may be spaced between 1 μm to 5 μm, or between 1 μm to 3 μm from the distal tip of the first electrode. For example, the distal tip of the second electrode may be about 1 μm, about 2 μm, about 3 μm, about 4 μm, or about 5 μm from the distal tip of the first electrode. The first and second electrodes can each be a hollow glass micropipette filled with a conductive liquid. An external alternating electric field E3 is applied to the cell, and a voltage between the first electrode 31 and the second electrode 32 is then measured while the external alternating electric field E3 is being applied. The external electric field can have a frequency between 50 kHz and 1 MHz or between 75 kHz and 500 kHz. This approach differs from the approach described above in connection with FIGS. 2 and 3 because the electrode spacing and positioning in the FIG. 5 approach is critical.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A method of determining a frequency for treating a particular subject using an alternating electric field, the method comprising:
electrically connecting a first electrode to an interior of a first cancer cell obtained from the subject;
electrically connecting a second electrode to an interior of a second cancer cell obtained from the subject;
electrically connecting a third electrode to the interior of the first cancer cell;
electrically connecting a fourth electrode to the interior of the second cancer cell;
applying an external alternating electric field to the first and second cancer cells at a plurality of different frequencies at a respective plurality of different times;
measuring a respective first voltage between the third electrode and the fourth electrode while the external alternating electric field is being applied at each of the plurality of different frequencies during times when a low resistance path is established between the first electrode and the second electrode;
measuring a respective second voltage between the third electrode and the fourth electrode while the external alternating electric field is being applied at each of the plurality of different frequencies during times when a low resistance path is not established between the first electrode and the second electrode; and
determining a frequency for treating the subject using an alternating electric field based at least in part on the measured first voltages and the measured second voltages.

2. The method of claim 1, wherein the determining comprises selecting a frequency at which a difference between the respective first voltage and the respective second voltage is largest.

3. The method of claim 1, wherein the determining comprises selecting a frequency at which a difference between the respective first voltage and the respective second voltage is within 10% of the largest difference.

4. The method of claim 1, wherein the determining comprises selecting a frequency at which a difference between the respective first voltage and the respective second voltage is within 25% of the largest difference.

5. The method of claim 1, wherein each of the plurality of different frequencies is between 50 kHz and 1 MHz.

6. The method of claim 1, wherein each of the plurality of different frequencies is between 75 kHz and 500 kHz.

7. The method of claim 1, wherein each of the plurality of different frequencies is between 75 kHz and 500 kHz, and
wherein the determining comprises selecting a frequency at which a difference between the respective first voltage and the respective second voltage is within 25% of the largest difference.

8. The method of claim 1, wherein the first electrode comprises a hollow glass micropipette filled with a conductive liquid, and wherein the second electrode comprises a hollow glass micropipette filled with a conductive liquid.

9. The method of claim 1, further comprising:
obtaining the first cancer cell from the subject;
obtaining the second cancer cell from the subject; and
treating the subject using an alternating electric field at the determined frequency.

10. An apparatus for determining a frequency for treating a particular subject using an alternating electric field, the apparatus comprising:
a first electrode configured to electrically connect with an interior of a first cell;
a second electrode configured to electrically connect with an interior of a second cell; and
a switch having a first terminal and a second terminal, wherein the first terminal is electrically connected to the first electrode and the second terminal is electrically connected to the second electrode, and wherein the switch can operate in either (a) a closed state that establishes a low resistance path between the first electrode and the second electrode or (b) an open state that does not establish a low resistance path between the first electrode and the second electrode.

11. The apparatus of claim 10, wherein the first electrode comprises a hollow glass micropipette filled with a conductive liquid, and wherein the second electrode comprises a hollow glass micropipette filled with a conductive liquid.

12. The apparatus of claim 10, further comprising:
the first cell, wherein the first electrode is electrically connected with the interior of the first cell; and
the second cell, wherein the second electrode is electrically connected with the interior of the second cell.

13. The apparatus of claim 12, further comprising:
a third electrode configured to electrically connect with the interior of the first cell, wherein the third electrode is electrically connected with the interior of the first cell; and
a fourth electrode configured to electrically connect with the interior of the second cell, wherein the fourth electrode is electrically connected with the interior of the second cell.

14. The apparatus of claim 13, further comprising a controller configured to
control an electric field generator and the state of the switch so that while the switch is in the closed state, the electric field generator applies an electric field at a plurality of different frequencies to the first cell and to the second cell at respective different first times, wherein each of the plurality of different frequencies is between 50 kHz and 1 MHz,
input a respective first voltage measurement obtained using the third electrode and the fourth electrode during each of the first times,
control the electric field generator and the state of the switch so that while the switch is in the open state, the electric field generator applies an electric field at the plurality of different frequencies to the first cell and to the second cell at respective different second times, and
input a respective second voltage measurement obtained using the third electrode and the fourth electrode during each of the second times.

15. The apparatus of claim 14, wherein the controller is further configured to determine a frequency that maximizes a difference between a respective first voltage measurement and a respective second voltage measurement.

16. A method of determining a frequency of an alternating electric field that will maximize an electric gradient when cells within a given population divide, the method comprising:
electrically connecting a first electrode to an interior of a first cell obtained from the population;
electrically connecting a second electrode to an interior of a second cell obtained from the population;
electrically connecting a third electrode to the interior of the first cell;
electrically connecting a fourth electrode to the interior of the second cell;
applying an external alternating electric field to the first and second cells at a plurality of different frequencies at a respective plurality of different times;
measuring a respective first voltage between the third electrode and the fourth electrode while the external alternating electric field is being applied at each of the plurality of different frequencies during times when a low resistance path is established between the first electrode and the second electrode;
measuring a respective second voltage between the third electrode and the fourth electrode while the external alternating electric field is being applied at each of the plurality of different frequencies during times when a low resistance path is not established between the first electrode and the second electrode; and
determining a frequency for applying an alternating electric field to the population of cells based at least in part on the measured first voltages and the measured second voltages.

17. The method of claim 16, wherein the determining comprises selecting a frequency at which a difference between the respective first voltage and the respective second voltage is largest.

18. The method of claim 16, wherein the determining comprises selecting a frequency at which a difference between the respective first voltage and the respective second voltage is within 10% of the largest difference.

19. The method of claim 16, wherein the determining comprises selecting a frequency at which a difference between the respective first voltage and the respective second voltage is within 25% of the largest difference.

20. The method of claim 16, wherein each of the plurality of different frequencies is between 75 kHz and 500 kHz, and
wherein the determining comprises selecting a frequency at which a difference between the respective first voltage and the respective second voltage is within 25% of the largest difference.
